SEQUENCE LISTING (3 sequences)

SEQ ID NO: 1 - protein, 20 aa, Artificial Sequence
  Synthetic Construct
ACGGTCAATAAGACCAGTAA

SEQ ID NO: 2 - protein, 21 aa, Artificial Sequence
  Synthetic Construct
CTGGGTTGTTTCAATCTAACA

SEQ ID NO: 3 - protein, 21 aa, Artificial Sequence
  Synthetic Construct
TCCCAATGAGTTGCCATATAA

(19) United States
(12) Patent Application Publication
(10) Pub. No.: US 2011/0288032 A1
(43) Pub. Date: Nov. 24, 2011

(54) ENZASTAURIN FOR THE TREATMENT OF CANCER

(75) Inventor: Gopinath Ganji, London (CA)

(73) Assignee: ELI LILLY AND COMPANY, Indianapolis, IN (US)

(21) Appl. No.: 13/130,104

(22) PCT Filed: Dec. 7, 2009
(86) PCT No.: PCT/US09/66925

§ 371 Date: May 19, 2011

Related U.S. Application Data
(60) Provisional application No. 61/122,451, filed on Dec. 15, 2008.

(51) Int. Cl. A61K 31/4545; A61K 38/12; A61K 31/506; A61P 35/00; A61K 38/15

(52) U.S. Cl. 514/19.4; 514/318; 514/19.3; 514/19.5; 514/275; 514/252.14

(57) ABSTRACT

The present invention relates to HDAC2 as a biological marker for treating cancer in a patient using Enzastaurin as a single agent or in combination with a Class I selective HDAC inhibitor.

The present invention relates to methods of using HDAC2 as a biological marker in conjunction with the treatment of cancer using Enzastaurin. The present invention also relates to the use of Enzastaurin in combination with a Class I selective HDAC inhibitor in order to achieve an enhanced therapeutic effect in treating cancer.

Enzastaurin is a PKC Beta selective inhibitor. Enzastaurin has the chemical name 3-(1-methyl-1H-indol-3-yl)-4-[1-[1-(pyridin-2-ylmethyl)piperidin-4-yl]-1H-indol-3-yl]-1H-pyrrole-2,5-dione and is disclosed in U.S. Pat. No. 5,668,152.

HDACs belong to the histone deacetylase superfamily. There are at least 18 HDAC enzymes which are categorized into 4 classes, based on their homology to yeast deacetylases. HDACs remove the acetyl group added by histone acetyltransferases. The removal of the acetyl group enables histones to bind to the DNA, restricting access to the DNA. Consequently, HDACs prevent transcription to occur.

Ropero reports that endometrial, colon and gastric tumor samples harbor HDAC2 inactivating mutations. Ropero, S., et al. (2006) Nat Genet, 38(5): 566-569. QRT-PCR on cancer cell lines and tumors (breast, glioblastomas, ovarian, renal, bladder, and colorectal tumors) have exhibited decreased levels of HDAC2 RNA. Ozdag, H., et al. (2006) BMC Genomics, 7: 90. Additionally, the ProteinAtlas (http://www.proteinatlas.org) reveals that moderate to negative immunohistochemistry (IHC) staining of HDAC2 is observed in subsets of gastric, endometrial, ovarian, breast, renal, cervical, liver, lung, malignant carcinoid, lymphoma, pancreatic, thyroid, and prostate tumors.

Class I HDACs are well-known transcriptional corepressors and always associate with transcriptional factors and cofactors in vivo. Biological data suggest that Class I HDACs are associated with cell cycle progression, metastasis, and apoptosis and are promising targets for cancer therapy. “Class 1 HDAC inhibitors,” such as, vorinostat, depsipeptide, MS-275, MGCD0103, belinostat, Baceca, panobinostat, PCI-24781, TSA, LAQ834, SBHA, Sodium butyrate, Valproic acid, Apicidin, Phenyl butyrate, CI994, Trapoxin, SB-429201, Bispyridinum diene, SHI-1:2, R306465, SB-379278A, and PCI-34051, are known.

Although much progress has been made toward understanding the biological basis of cancer and in its treatment, it is still one of the leading causes of death. Variations in patient response to drugs pose a significant challenge as resistance and lack of response are commonly encountered in the clinic. Many factors are thought to play roles in the variations in patient responses to drugs including genetics, concomitant drug therapies, environment, lifestyle, health status, and disease status.

A medical need exists to identify patients that will best respond to chemotherapy regimens. Few predictive biological markers have been identified and fewer developed into diagnostic tests to definitively guide treatment decisions. A patient selection approach is of significant value to tailor the use of Enzastaurin in treating cancer. It would be of great value to have methods to timely determine if a patient will likely respond to treatment with Enzastaurin.

The present invention relates to methods of treating cancer with Enzastaurin after first determining the expression level of HDAC2, which can be used as a biological marker of Enzastaurin efficacy. When the level of HDAC2 is low or undetectable, Enzastaurin alone is expected to be particularly effective. When the level of HDAC2 is high, the invention involves administering an effective amount of Enzastaurin in combination with a Class I selective HDAC inhibitor.

The present invention includes a method of treating cancer in a patient, comprising administering an effective amount of Enzastaurin to the patient wherein the patient has a low or undetectable level of HDAC2.

Furthermore, the present invention provides a method of treating cancer in a patient, comprising: a) obtaining a sample comprising cancer cells from the patient; b) determining the level of HDAC2 in the cancer sample; and c) administering an effective amount of Enzastaurin to the patient if the cancer sample has a low or undetectable level of HDAC2.

The present invention includes a method of treating cancer in a patient, comprising administering an effective amount of Enzastaurin to the patient wherein the patient has a HDAC2 frameshift nonsense mutation.

Additionally, the present invention provides a method of treating cancer in a patient, comprising: a) obtaining a sample comprising cancer cells from the patient; b) determining whether HDAC2 is mutated in the cancer sample; and c) administering an effective amount of Enzastaurin to the patient if the patient sample has a HDAC2 frameshift nonsense mutation.

The present invention includes a method of treating cancer in a patient, comprising administering an effective amount of Enzastaurin and an effective amount of a Class I selective HDAC inhibitor to the patient wherein the patient has a high level of HDAC2.

Furthermore, the present invention provides a method of treating cancer in a patient, comprising: a) obtaining a sample comprising cancer cells from the patient; b) determining the level of HDAC2 in the cancer sample; and c) administering an effective amount of Enzastaurin and an effective amount of a Class I selective HDAC inhibitor to the patient if the cancer sample has a high level of HDAC2.

The present invention includes the use of Enzastaurin in the manufacture of a medicament for treating cancer in a patient, wherein the patient has a low or undetectable level of HDAC2.

Furthermore, the present invention provides the use of Enzastaurin in combination with a Class I selective HDAC inhibitor in the manufacture of a medicament for treating cancer in a patient, wherein the patient has a high level of HDAC2, and wherein said medicament is to be administered in combination with a Class I selective HDAC inhibitor.

The present invention provides methods and uses as described herein, in which the cancer is selected from the group consisting of colorectal cancer, gastric cancer, endometrial cancer, ovarian cancer, breast cancer, liver cancer, lung cancer, renal cancer, cutaneous T-cell lymphoma, glioblastoma, lymphoma, pancreatic cancer, and prostate cancer. Furthermore, the Class I selective HDAC inhibitor may be selected from the group consisting of vorinostat, depsipeptide, MS-275, MGCD0103, belinostat, Baceca, panobinostat, PCI-24781, TSA, LAQ834, SBHA, Sodium butyrate, Valproic acid, Apicidin, Phenyl butyrate, CI994, Trapoxin, SB-429201, Bispyridinum diene, SHI-1:2, R306465, SB-379278A, and PCI-34051.

The present invention includes the identification of biological markers to aid in the prediction of patient outcome and the informed selection of currently available therapies for the use of Enzastaurin in cancer treatment. The present invention employs HDAC2 as the preferred biological marker.

The genetic aberrations acquired during the development of tumors represent both the drivers of disease and the opportunities for tailored therapeutics in cancer. Patients with genes and pathways altered in specific tumor types may respond differently to targeted therapies. Understanding these genetic determinants of drug sensitivity early in the discovery process can help to improve and accelerate decisions regarding clinical indications, patient stratification, and combination studies.

These subpopulations represent patient groups with a compromised HDAC2 profile that can be targeted to improve therapeutic benefit and response to Enzastaurin as a single agent or in combination with a Class I selective HDAC inhibitor.

The present invention relates to treating a cancer that is selected from the group consisting of colorectal cancer, gastric cancer, endometrial cancer, ovarian cancer, breast cancer, liver cancer, lung cancer, renal cancer, cutaneous T-cell lymphoma, glioblastoma, lymphoma, pancreatic cancer, and prostate cancer.

The present invention provides for the use of Class I selective HDAC inhibitors that are selected from the group consisting of vorinostat, depsipeptide, MS-275, MGCD0103, belinostat, Baceca, panobinostat, PCI-24781, TSA, LAQ834, SBHA, Sodium butyrate, Valproic acid, Apicidin, Phenyl butyrate, CI994, Trapoxin, SB-429201, Bispyridinum diene, SHI-1:2, R306465, SB-379278A, and PCI-34051 in combination with Enzasturin.

Many methods are known to determine gene or protein expression in a cancer cell. Immunohistochemistry, Western blots, microarrays, and polymerase chain reaction (PCR) are a few examples that have been used to gain a molecular understanding of cancer types, subtypes, prognosis, and treatment effects. The development of these methods for the measurement of gene and protein expression makes it possible to search and systematically evaluate biological markers of cancer classification and outcome prediction in a variety of tumor types.

In the present invention, HDAC2 protein expression is preferably assayed or detected by Western blot or immunohistochemistry. Furthermore, in the present invention, the HDAC2 mutation is assayed by polymerase chain reaction (PCR) followed by sequencing to determine if the mutant allele is present. The detection method employed will change based on the availability of expertise, technology, and reagents.

The following definitions are provided to aid those of ordinary skill in the art in understanding the disclosure herein. These definitions are intended to be representative of those known in the art, and are therefore not limited to the specific elements presented.

The term “treating” (or “treat” or “treatment”) refers to the process involving a slowing, interrupting, arresting, controlling, reducing, or reversing the progression or severity of a symptom, disorder, condition, or disease.

A “patient” is a mammal, preferably a human.

The term “effective amount” refers to the amount or dose of Enzastaurin or HDAC2 inhibitor or pharmaceutically acceptable salt, upon which single or multiple dose administration to a patient, provides the desired treatment. In general, optimum dosages of each of these therapeutic agents can vary depending on the relative potency of the active ingredients in individual patients. Medical practitioners can determine dose and repetition rates for dosing based on measured residence times and concentrations of the active ingredients in bodily fluids or tissues and/or monitoring of relevant disease-related biomarkers for particular cancers.

The term “detectable level” refers to the gene, gene transcript, or gene product being present at a level that is detected in a biological sample by a diagnostic method or assay, such as Western blot or immunohistochemistry. In the present invention, low or undetectable level of HDAC2 refers to <20% expression of HDAC2 by Western blot relative to the HDAC2 expression in HCT116 cells. Furthermore, in the present invention, high level of HDAC2 refers to >20% expression of HDAC2 by Western blot relative to the HDAC2 expression in HCT116 cells.

HDAC2 expression can be measured in a sample using techniques well established in the art. Essentially, tumor biopsies are taken from a patient. Tissues are homogenized and lysates are analyzed by Western blot to determine the amount of HDAC2 protein expression. In case of formalin fixed paraffin embedded (FFPE) samples, tumor cores are sectioned and stained for HDAC2 detection by immunohistochemistry. A histopathologist scores these samples as low or high by an immunohistochemistry scoring method known, such as an H-score.

The term “frameshift nonsense mutation” refers to the truncating or inactivating mutation in the HDAC2 gene as reported. Ropero, S., et al. (2006) Nat Genet, 38(5): 566-9.

The frameshift nonsense mutation can be determined by using well established methods. Basically, DNA from a patient sample is analyzed by polymerase chain reaction (PCR) and direct sequencing to determine the presence of a frameshift mutation. The sequence chromatograms obtained from the DNA sample is compared to the wild type sequence to look for a truncating mutation. Ropero, S., et al.

### EXAMPLE 1

### HDAC2 as a Sensitizer of Enzastaurin Drug Response

HCT116 cells are obtained from American Tissue Culture Collection, ATCC (Rockville, Md., USA) and cultured in McCoy's 5A medium supplemented with 2 mM L-glutamine and 10% fetal bovine serum (FBS), in a humidified 37° C. incubator with 5% CO₂. Plates (384-well) are pre-printed using 2 siRNAs per target in the Druggable Genome v2 Library (Qiagen) such that each well contains 13 nM of an individual siRNA duplex. High throughput reverse transfections are performed by adding transfection agent Lipofectamine 2000 (Invitrogen) and 1500 cells diluted in McCoy's 5A medium supplemented with 2 mM L-glutamine and 2% FBS into each well following a standard reverse transfection protocol. Twenty-four hours post transfection, each assay plate is treated with or without 5 concentrations (0-10 μM) of Enzastaurin in 1% DMSO. Seventy-two hours later, cell viability is measured using chemiluminescence based CellTiter Glo (Promega) assay readout, according to manufacturer's recommendations. UBB siRNA (Qiagen) is the positive cell killing control and All Star Non-silencing (NS-AS) or green fluorescent protein (GFP) is the negative control.

Raw signal values are normalized to untreated control wells to compare across plates. These are fit to a 4-parameter logistical model to determine IC50 values. A ‘shift’ in IC50 with respect to the negative control (described above) is calculated as: (IC50 target−IC50 control) divided by IC50 control.

For RT-PCR, cells are reverse transfected as described above and incubated with siRNAs for 72 hours at 37° C. and washed with 1×PBS using a plate washer before lysis. RNA is extracted using magnetic beads (Ambion, MagMax-96 Total RNA Isolation Kit, Cat #1830) according to the manufacturer's protocol. Total RNA concentration of the samples is measured using a NanoDrop-1000 spectrophotometer. Bio-Rad's iScript cDNA Synthesis Kit (Cat #170-8891) is used for cDNA synthesis and reactions are run on MJ Research's DNA Engine Tetrad Peltier Thermal Cycler according to the manufacturer's recommendation. Five nanograms (5 ng) of cDNA are used per 10 μL qPCR reaction volume. Gene-specific qPCR is conducted using TaqMan® probe chemistry (ABI, Foster City, Calif.) and run on an ABI 7900HT Fast Real-time PCR System. The reactions are carried out in triplicate per sample with endogenous glyceraldehyde-3-phosphate dehydrogenase (GAPDH), buffer, scrambled (described above) and non-template (a standard for the probe) controls. Gene expression values are normalized to GAPDH and calculated by the relative quantification method (ΔΔC_{T} method) using ABI's SDS RQ Manager 1.2 software. The C_{T} is a standard metric, which refers to the cycle threshold number. Knockdown of a gene of interest by a particular siRNA relative to endogenous expression is given by:

(ΔC_{T})test=[Average Target Gene C_{T}−Average GAPDH C_{T}]test

(ΔC_{T})control=[Average Target Gene C_{T}−Average GAPDH C_{T}]control

ΔΔC_{T}=(ΔC_{T})test−(ΔC_{T})Control

RQ=2^{-ΔΔC}^{_{T}}

%KD=(RQsi−RQbuffer)*100/RQbuffer

where ‘test’ refers to siRNA treated (si) or buffer control (buffer); ‘control’ refers to scrambled siRNA control, a negative control. RQsi and RQbuffer are calculated as shown above to determine relative gene expression values for a target of interest with and without (endogenous levels) siRNA treatment, respectively.

Three siRNAs that target HDAC2 cause a shift in dose response kill curve relative to negative control as seen by >2 fold shift in IC50 values, sensitizing HCT116 to the effects of Enzastaurin. High content images also reflect a higher degree of cell killing in HCT116 cells treated with Enzastaurin and HDAC2 siRNA relative to negative controls and either condition alone (Data not shown).

| | Enzastaurin | | |
|---|---|---|---|
| HDAC2 siRNA Sequence | IC50 (μM) | Shift | % KD |
| ACGGTCAATAAGACCAGTAA | 1.36 | 0.63 | 96 |
| (SEQ ID NO: 1) | | | |
| CTGGGTTGTTTCAATCTAACA | 1.68 | 0.54 | 95 |
| (SEQ ID NO: 2) | | | |
| TCCCAATGAGTTGCCATATAA | 2.01 | 0.51 | 91 |
| (SEQ ID NO: 3) | | | |
| None | 3.69 | 0 | 0 |

### EXAMPLE 2

### Enhanced Activity of Enzastaurin in RKO Relative to HCT116

The human colon cancer cell line, HCT116 (HDAC 2 w.t), and RKO (HDAC 2+/−), a cell line containing a nonsense mutation resulting in null protein expression of HDAC2 relative to HCT116, are obtained from American Tissue Culture Collection, ATCC (Rockville, Md., USA) and cultured in the ATCC recommended growth medium supplemented with 2 mM L-glutamine and 10% FBS, in a humidified 37° C. incubator with 5% CO₂. Drug dose response experiments are performed by seeding 1000-2000 cells diluted in McCoy's 5A medium containing 25 mM N-2-hydroxyethylpiperazine-N′-2-ethanesulfonic acid (HEPES), 2 mM L-glutamine and 2% fetal bovine serum (FBS) followed by treatment with or without serial dilutions of Enzastaurin (0-100 μM) in 1% DMSO. Seventy-two or ninety-six hours later, cell viability is measured using chemiluminescence based CellTiter Glo (Promega) assay readout, according to manufacturer's recommendations. Raw signal values are normalized to untreated control and analyzed by non-linear curve fitting in GraphPad Prism (La Jolla, Calif., USA).

Drug dose response curves show significant differences (>2×) in IC50 and maximum effect of growth inhibition by Enzastaurin in RKO cells relative to HCT116. The IC50 of Enzastaurin in HCT116 cells is 8.34 μM compared to an IC50 of 3.56 μM in RKO cells. Furthermore, the maximum killing effect of Enzastaurin in RKO (95-100%) is greater than that of HCT116 (50-60%). These data provide genetic confirmation of HDAC2 knockdown as a sensitizer to Enzastaurin response.

### EXAMPLE 3

### In Vitro Growth Inhibition and Combination Drug Studies

To determine whether a Class I selective HDAC inhibitor and Enzastaurin provide a beneficial effect, MS-275, a Class I selective HDAC inhibitor, and Enzastaurin are assayed for cancer cell growth inhibition.

Human colon cancer cell line HCT116 obtained from American Tissue Culture Collection, ATCC (Rockville, Md., USA) is maintained as monolayer in McCoy's 5A medium containing 25 mM HEPES, 2 mM L-glutamine and 10% FBS, in a humidified 37° C. incubator with 5% CO₂. Exponentially growing HCT116 cells (2000 cells/well) are plated in Poly-D-Lysine coated 96-well plates in McCoy's 5A medium containing 25 mM HEPES, 2 mM L-glutamine and 2% FBS for 24 h prior to drug treatment. Cells are treated for 72 hours with (i) a range of concentrations of Enzastaurin (0-10 μM) and MS-275 (0-4 μM) alone to determine IC50 values from sigmoidal dose responsive curves (ii) concurrent addition of Enzastaurin and MS-275 at 3 fixed IC50 ratios (2.5, 5, 10), all in a final DMSO concentration of 0.02% following a fixed ratio design (Koizumi, F., et al. (2004) Int J Cancer, 108(3): 464-72; Tallarida, R. J., et al. (1997) Life Sci, 61(26): PL 417-25). Cells are then fixed and stained with Propidium iodide (PI). Cell counts are measured by the Acumen Explorer system (Acumen Bioscience Ltd, UK).

Data analysis is performed by the median effect principle suggested by Chou and Talalay (Chou, T. C. and P. Talalay, Quantitative analysis of dose-effect relationships: the combined effects of multiple drugs or enzyme inhibitors, Adv Enzyme Regul, 1984. 22: p. 27-55) by using the Calcusyn software (Biosoft, Cambridge, UK) to calculate a Combination Index (CI). CI is a quantitative measure of the degree of interaction between different drugs: CI=1 for additivity; CI>1 for antagonism; and CI<1 for synergism. In the table below, Fa is the Fraction affected; CI is the combination index; SD is the standard deviation; E means Enzastaurin; M means MS-275; E/M means the fixed ratio of each drug's IC50 values.

| | E/M = 2.5 | E/M = 5 | E/M = 10 |
|---|---|---|---|
| Fa | CI ± SD | CI ± SD | CI ± SD |
| 0.5 | 0.883 ± 0.1288 | 0.639 ± 0.0681 | 0.566 ± 0.0537 |
| 0.6 | 0.838 ± 0.1117 | 0.603 ± 0.0602 | 0.529 ±.0.0487 |
| 0.7 | 0.791 ± 0.0989 | 0.567 ± 0.0552 | 0.492 ± 0.0462 |
| 0.8 | 0.738 ± 0.0914 | 0.525 ± 0.0538 | 0.450 ± 0.0462 |
| 0.9 | 0.664 ± 0.0932 | 0.469 ± 0.0577 | 0.393 ± 0.0496 |
| 0.99 | 0.487 ± 0.1253 | 0.335 ± 0.0758 | 0.265 ± 0.0599 |

Simultaneous combination drug studies of Enzastaurin and MS-275 demonstrate synergistic interaction (CI<1) across all fixed ratios and Fa values tested. These data provide pharmacological evidence for HDAC2 depletion enhancing Enzastaurin action.

We claim:

1. A method of treating cancer in a patient, comprising administering an effective amount of Enzastaurin to the patient wherein the patient has a low or undetectable level of HDAC2.

2. A method of treating cancer in a patient, comprising:
a) obtaining a sample comprising cancer cells from the patient;
b) determining the level of HDAC2 in the cancer sample; and
c) administering an effective amount of Enzastaurin to the patient if the patient sample has a low or undetectable level of HDAC2.

3. A method of treating cancer in a patient, comprising administering an effective amount of Enzastaurin to the patient wherein the patient has a HDAC2 frameshift nonsense mutation.

4. A method of treating cancer in a patient, comprising:
a) obtaining a sample comprising cancer cells from the patient;
b) determining whether HDAC2 is mutated in the cancer sample; and
c) administering an effective amount of Enzastaurin to the patient if the patient sample has a HDAC2 frameshift nonsense mutation.

5. A method of treating cancer in a patient, comprising administering an effective amount of Enzastaurin and an effective amount of Class I selective HDAC inhibitor to the patient wherein the patient has a high level of HDAC2.

6. A method of treating cancer in a patient, comprising:
a) obtaining a sample comprising cancer cells from the patient;
b) determining the level of HDAC2 in the cancer sample; and
c) administering an effective amount of Enzastaurin and an effective amount of Class I selective HDAC inhibitor to the patient if the patient sample has a high level of HDAC2.

7. The method of either claim 5 or 6, wherein the Class I selective HDAC inhibitor is selected from the group consisting of vorinostat, depsipeptide, MS-275, MGCD0103, belinostat, Baceca, panobinostat, PCI-24781, TSA, LAQ834, SBHA, Sodium butyrate, Valproic acid, Apicidin, Phenyl butyrate, CI994, Trapoxin, SB-429201, Bispyridinum diene, SHI-1:2, R306465, SB-379278A, and PCI-34051.

8. The method of any one of claims 1-7, wherein the cancer is selected from the group consisting of colorectal cancer, gastric cancer, endometrial cancer, ovarian cancer, breast cancer, liver cancer, lung cancer, renal cancer, cutaneous T-cell lymphoma, glioblastoma, lymphoma, pancreatic cancer, and prostate cancer.

9. Use of Enzastaurin in the manufacture of a medicament for treating cancer in a patient, wherein the patient has a low or undetectable level of HDAC2.

10. Use of Enzastaurin in combination with a Class I selective HDAC inhibitor in the manufacture of a medicament for treating cancer in a patient, wherein the patient has a high level of HDAC2, and wherein said medicament is to be administered in combination with a Class I selective HDAC inhibitor.

11. The use of claim 10, wherein the Class I selective HDAC inhibitor is selected from the group consisting of vorinostat, depsipeptide, MS-275, MGCD0103, belinostat, Baceca, panobinostat, PCI-24781, TSA, LAQ834, SBHA, Sodium butyrate, Valproic acid, Apicidin, Phenyl butyrate, CI994, Trapoxin, SB-429201, Bispyridinum diene, SHI-1:2, R306465, SB-379278A, and PCI-34051.

12. The use of any one of claims 9-11, wherein the cancer is selected from the group consisting of colorectal cancer, gastric cancer, endometrial cancer, ovarian cancer, breast cancer, liver cancer, lung cancer, renal cancer, cutaneous T-cell lymphoma, glioblastoma, lymphoma, pancreatic cancer, and prostate cancer.
